(19) United States
(12) Patent Application Publication
(10) Pub. No.: US 2025/0032247 A1
(43) Pub. Date: Jan. 30, 2025

(54) Stent-Matched Radiopaque Marker

(71) Applicant: St. Jude Medical, Cardiology Division, Inc., St. Paul, MN (US)

(72) Inventors: Alec King, Maple Grove, MN (US); Somnhot Thamlansy, New Hope, MN (US)

(73) Assignee: St. Jude Medical, Cardiology Division, Inc., St. Paul, MN (US)

(21) Appl. No.: 18/777,898

(22) Filed: Jul. 19, 2024

Related U.S. Application Data
(60) Provisional application No. 63/515,180, filed on Jul. 24, 2023.

(51) Int. Cl. A61F 2/24

(52) U.S. Cl. CPC A61F 2/2418; A61F 2/243; A61F 2220/0075; A61F 2250/0098

(57) ABSTRACT

A prosthetic heart valve may include an expandable frame with an anchoring section and a valve support section. The anchoring section may form a row of cells, two adjacent cells being joined together by a runner extending in a longitudinal direction. The anchoring section may include a plurality of commissure attachment features (“CAFs”), a first one of the CAFs features being axially aligned with the runner. Prosthetic leaflets may be mounted within the frame. A first pair of the plurality of prosthetic leaflets may be coupled together to form a first prosthetic commissure attached to the first one of the CAFs. A radiopaque marker may have a longitudinal section extending between a first end section and a second end section, and be coupled to the runner so that the longitudinal section is axially aligned with both the runner and the first one of the plurality of commissure attachment features.

### CROSS-REFERENCE TO RELATED APPLICATIONS

This application claims priority to the filing date of U.S. Provisional Patent Application No. 63/515,180, filed Jul. 24, 2023, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement or implantation (“TAVR” or “TAVI”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to as a valve assembly) mounted to/within an expandable stent (the terms “stent” and “frame” are used interchangeably herein). In general, these collapsible/expandable heart valves include a self-expanding or balloon-expandable stent, often made of nitinol or another shape-memory metal or metal alloy (for self-expanding stents) or a plastically expandable materials such as steel or cobalt chromium (for balloon-expandable stents). Existing collapsible/expandable TAVR devices have been known to use different configurations of stent layouts-including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

When expanding a prosthetic heart valve into the native heart valve annulus, accurate deployment is typically an important indicator of the success of the prosthesis. For example, for an aortic heart valve replacement, the position of the prosthesis relative to the aortic annulus, as well as the extent to which the prosthesis extends into the left ventricular outflow tract (“LVOT”), can impact performance attributes of the prosthesis such as hemodynamics, PV leak, and the necessity of implanting a pacemaker with the prosthetic heart valve. Similarly, the rotational position of the prosthesis relative to the anatomy may be important. For example, it may be desirable to rotationally align the commissures of the prosthetic leaflets with the commissures of the native valve leaflets in an aortic valve replacement. One reason for this is that, by aligning the prosthetic commissures with the native commissures, access to the coronary arteries may be better maintained for future procedures, such as coronary stent implantations. Thus, it would be desirable to be able to increase the accuracy with which the prosthetic heart valve can be placed within the native valve annulus to optimize performance attributes of the prosthetic heart valve.

### SUMMARY OF THE DISCLOSURE

According to one aspect of the disclosure, a prosthetic heart valve includes an expandable frame extending along a longitudinal axis between an inflow end and an outflow end. The frame may have an anchoring section adjacent to the inflow end and a valve support section adjacent to the anchoring section. The anchoring section may include struts forming a row of cells, two adjacent cells in the row of cells being joined together by a runner extending in a longitudinal direction parallel to the longitudinal axis. The anchoring section may include a plurality of commissure attachment features, a first one of the plurality of commissure attachment features being axially aligned with the runner. A plurality of prosthetic leaflets may be mounted within the frame. A first pair of the plurality of prosthetic leaflets may be coupled together to form a first prosthetic commissure, the first prosthetic commissure being attached to the first one of the plurality of commissure attachment features. A radiopaque marker may have a longitudinal section extending between a first end section and a second end section. The radiopaque marker may be coupled to the runner so that the longitudinal section is axially aligned with both the runner and the first one of the plurality of commissure attachment features. The radiopaque marker may be non-integral with the frame.

The row of cells may be an inflow-most row of cells. The anchoring section may include a second row of cells adjacent to the inflow-most row of cells. The plurality of prosthetic leaflets may comprise exactly three prosthetic leaflets, and the plurality of commissure attachment features may comprise exactly three commissure attachment features. A second pair of the plurality of prosthetic leaflets may be coupled together to form a second prosthetic commissure attached to a second one of the plurality of commissure attachment features, and a third pair of the plurality of prosthetic leaflets are coupled together to form a third prosthetic commissure attached to a third one of the plurality of commissure attachment features. The radiopaque marker may be the only radiopaque marker coupled to the prosthetic heart valve.

The prosthetic heart valve may include an inner cuff positioned between the plurality of prosthetic leaflets and the frame. A buffer may be positioned between the radiopaque marker and the runner. The buffer may be formed of polymer or fabric. The radiopaque marker may have two planes of symmetry, or fewer than two planes of symmetry. The frame may be formed of stainless steel or cobalt chromium, and the radiopaque marker may be formed of gold, platinum, or tantalum.

The radiopaque marker may include first and second angled protrusions extending from the first end section of the longitudinal section, and third and fourth angled protrusions extending from the second end section of the longitudinal section. The first, second, third, and fourth angled protrusions may be semicircular. Two upper struts may extend at angles from a top of the runner, and two lower struts may extend at angles from a bottom of the runner. In an expanded condition of the prosthetic heart valve, the angles at which the two upper struts extend from the top of the runner may be the same as angles at which the first and second angled protrusions extend from the first end section of the longitudinal section of the radiopaque marker. In the expanded condition of the prosthetic heart valve, the angles at which the two lower struts extend from the bottom of the runner may be the same as angles at which the third and fourth angled protrusions extend from the second end section of the longitudinal section of the radiopaque marker.

The radiopaque marker may be sutured to the frame with a suture pattern. The suture pattern may include a vertical suture member extending in a direction parallel to the longitudinal axis, the vertical suture member wrapping around the radiopaque marker and the runner. The suture pattern may include at least one horizontal suture member extending in a direction transverse the longitudinal axis, the at least one horizontal suture member wrapping around the radiopaque marker and the runner. At least one recess may be formed in an edge of the longitudinal section of the radiopaque marker, the at least one horizontal suture member being received within the at least one recess. The longitudinal section of the radiopaque marker may be defined at least in part by two lateral edges, and the runner may be defined at least in part by two lateral edges, the two lateral edges of the radiopaque marker being aligned with the two lateral edges of the runner. The radiopaque marker may include a plurality of radiopaque markers, each of the plurality of radiopaque markers having a unique shape.

According to another aspect of the disclosure, a prosthetic heart valve may include an expandable frame extending along a longitudinal axis between an inflow end and an outflow end. The frame may have a plurality of cells, and each of the plurality of cells may be formed at least by two inflow struts and two outflow struts. Two circumferentially adjacent cells may connect at a strut intersection member where two of the inflow struts of the two circumferentially adjacent cells connect to two of the outflow struts of the two circumferentially adjacent cells. The frame may include a plurality of commissure attachment features. A first one of the plurality of commissure attachment features may be axially aligned with the strut intersection member of the two circumferentially adjacent cells. A plurality of prosthetic leaflets may be mounted within the frame. A first pair of the plurality of prosthetic leaflets may be coupled together to form a first prosthetic commissure, and the first prosthetic commissure may be attached to the first one of the plurality of commissure attachment features. A radiopaque marker may have a first portion and a second portion. The first portion of the radiopaque marker may define a window. The second portion of the radiopaque marker may be coupled to the strut intersection member of the two circumferentially adjacent cells so that so that the window and the second portion is aligned with (i) the strut intersection member of the two circumferentially adjacent cells and (ii) the first one of the plurality of commissure attachment features. The radiopaque marker may be non-integral with the frame.

The plurality of cells may include a first inflow row of cells, a second middle row of cells, and a third outflow row of cells. The two circumferentially adjacent cells may be part of the second middle row of cells. In another embodiment, the plurality of cells may include a first inflow row of cells and a second outflow row of cells, and the two circumferentially adjacent cells are part of the first inflow row of cells. The plurality of prosthetic leaflets may comprise exactly three prosthetic leaflets, and the plurality of commissure attachment features may comprise exactly three commissure attachment features. A second pair of the plurality of prosthetic leaflets may be coupled together to form a second prosthetic commissure attached to a second one of the plurality of commissure attachment features, and a third pair of the plurality of prosthetic leaflets may be coupled together to form a third prosthetic commissure attached to a third one of the plurality of commissure attachment features. The radiopaque marker may be the only radiopaque marker coupled to the prosthetic heart valve.

The frame may be formed of stainless steel or cobalt chromium, and the radiopaque marker may be formed of gold, platinum, or tantalum. The window may be circular or tear drop-shaped, and in an expanded condition of the frame, the window may be radially aligned with an empty space defined by a selected one of the plurality of cells. The first portion of the radiopaque marker may have a first shape that at least partially matches a shape of the two of the outflow struts of the two circumferentially adjacent cells, and the second portion of the radiopaque marker may have a second shape that at least partially matches a shape of the two of the inflow struts of the two circumferentially adjacent cells.

The prosthetic heart valve may include an inner cuff on an interior surface of the frame, and an outer cuff on an exterior surface of the frame. The radiopaque marker may be positioned between the inner cuff and the outer cuff. The inner cuff and the outer cuff may each be formed of fabric. The second portion of the radiopaque marker may be coupled to the strut intersection member by one or more sutures having a suture pattern. The suture pattern may include a horizontal portion that wraps around the second portion of the radiopaque marker and the strut intersection member. The suture pattern may include a vertical portion that wraps around the second portion of the radiopaque marker and the strut intersection member, the vertical portion passing through the window.

According to another aspect of the disclosure, a method of implanting a prosthetic heart valve includes advancing the prosthetic heart valve through a vasculature of a patient while the prosthetic heart valve is in a collapsed condition until the prosthetic heart valve is positioned within or adjacent to a native heart valve annulus of the patient. The prosthetic heart valve may include a frame defining a plurality of cells and may be formed of a metal or metal alloy having a first density. A radiopaque marker may be coupled to the frame, the radiopaque marker having a second density greater than the first density. The method may include, while the prosthetic heart valve is positioned within or adjacent to the native heart valve annulus of the patient, viewing an image of the prosthetic heart valve positioned within or adjacent to the native heart valve annulus of the patient to determine a position of a target on the radiopaque marker relative to an anatomical landmark in the image. The prosthetic heart valve may be deployed by expanding the prosthetic heart valve into the native heart valve annulus. The step of deploying the prosthetic heart valve may be performed after determining that the target on the radiopaque marker is aligned with the anatomical landmark in the image. The target on the radiopaque marker may be selected from a plurality of targets on the radiopaque marker based on the expected diameter that the prosthetic heart valve will have after expanding the prosthetic heart valve into the native heart valve annulus. The radiopaque marker may include a first portion and a second portion, the first portion of the radiopaque marker defining a window, the second portion of the radiopaque marker being coupled to a portion of the frame. The plurality of targets on the radiopaque marker may include (i) a center of the window and (ii) another location within the window spaced from the center of the window. The anatomical landmark may be a plane of the native heart valve annulus. While viewing the image of the prosthetic heart valve, a portion or an entirety of the window may be unobstructed by the portion of the frame to which the second portion of the radiopaque marker is coupled.

### BRIEF DESCRIPTION OF THE DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIG. 2A illustrates a prosthetic heart valve crimped over a balloon of a delivery device.

FIG. 2B is a schematic view of the balloon of FIG. 2A after having been inflated.

FIG. 3 is a plan view of a portion of a frame according to another aspect of the disclosure, as if the frame has been cut longitudinally and laid flat on a table.

FIG. 4 is a front view of a radiopaque marker according to one aspect of the disclosure.

FIG. 5 is a perspective view of the radiopaque marker of FIG. 4 coupled to the frame of FIG. 3, with the stent being in an expanded condition.

FIG. 6A is a radiograph of a prosthetic heart valve incorporating the frame of FIG. 3 and radiopaque marker of FIG. 4, the prosthetic heart valve being in a collapsed condition.

FIG. 6B is a radiograph of the prosthetic heart valve of FIG. 6A in an expanded condition.

FIG. 7A is a plan view of an alternate embodiment of the radiopaque marker of FIG. 4.

FIG. 7B is a plan view of an alternate embodiment of the radiopaque marker of FIG. 4.

FIG. 8A is a plan view of another embodiment of a radiopaque marker.

FIG. 8B is plan view of a portion of a frame according to another aspect if the disclosure, as if the frame has been cut longitudinally and laid flat on a table, with the radiopaque marker of FIG. 8A coupled thereto.

FIG. 8C is an enlarged view of the radiopaque marker of FIG. 8A coupled to the frame of FIG. 8B.

FIG. 8D is a side view of the radiopaque marker of FIG. 8A coupled to the frame of FIG. 8B, with additional components of a prosthetic heart valve shown coupled to the frame.

FIG. 8E is a radiograph of the radiopaque marker of FIG. 8A coupled to the frame of FIG. 8B, with the radiograph showing the frame at a first rotational angle.

FIG. 8F is a radiograph of the radiopaque marker of FIG. 8A coupled to the frame of FIG. 8B, with the radiograph showing the frame at a second rotational angle different than the first rotational angle shown in FIG. 8E.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to the trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

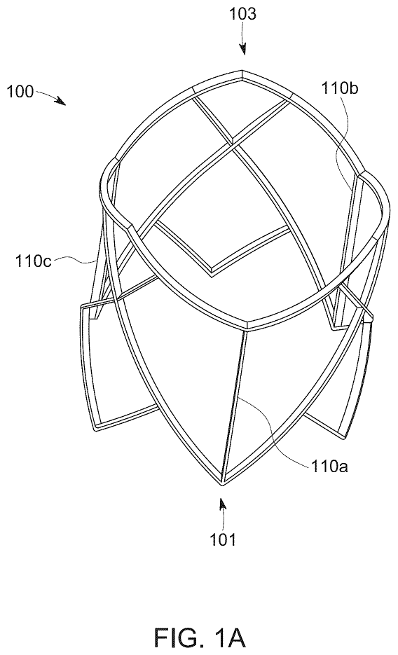

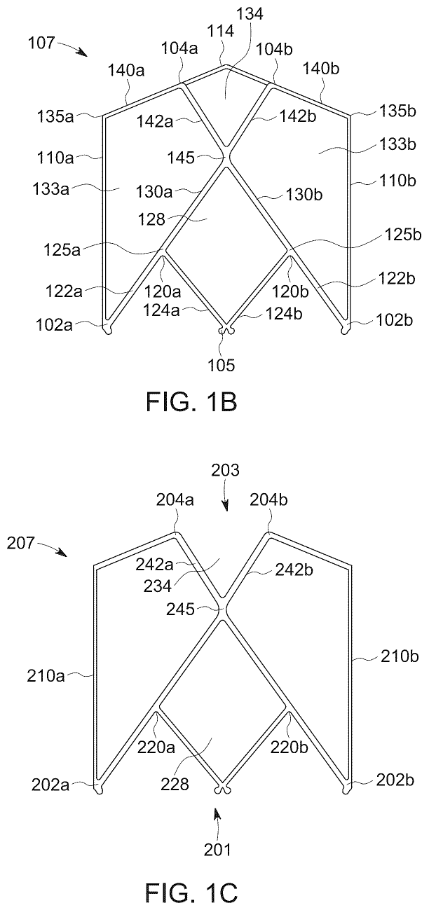

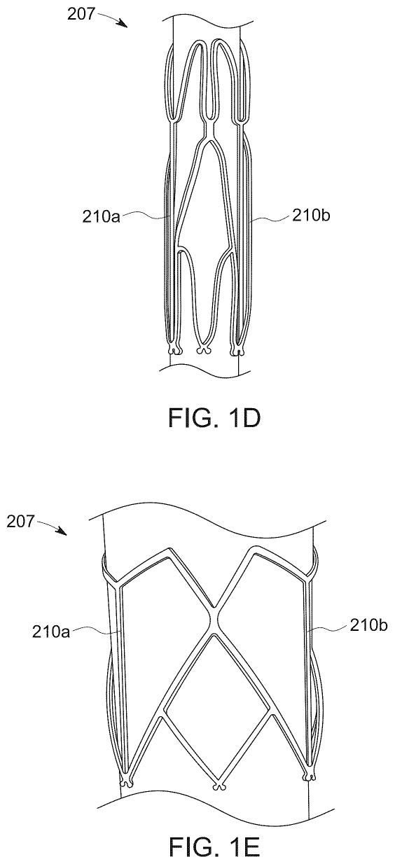

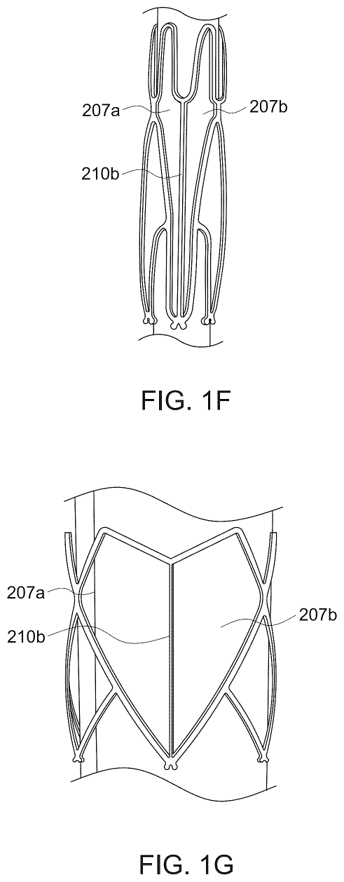

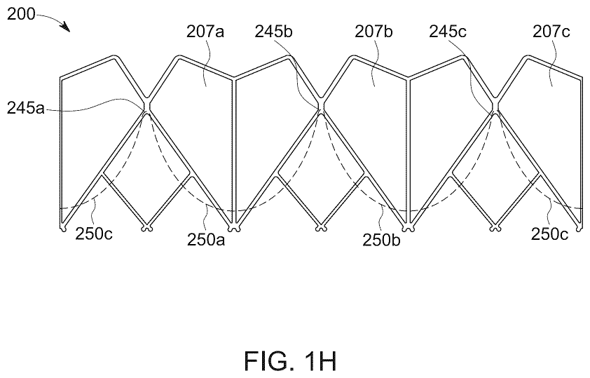

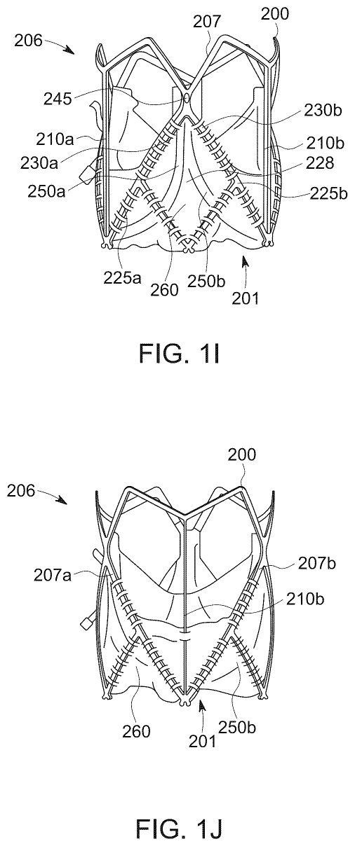

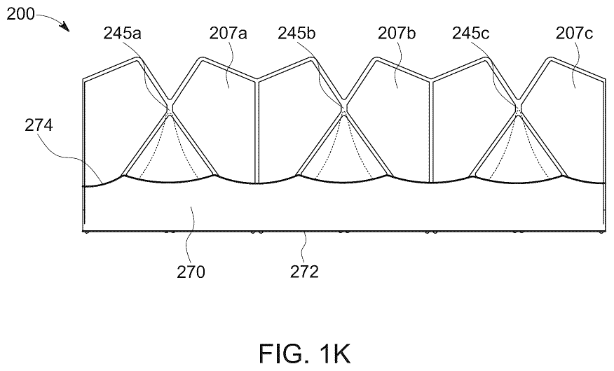

FIG. 1A illustrates a perspective view of a frame or stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100 and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail, and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135a and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as plastics, fabrics, and/or polymers, including ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 1I shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help minimize or eliminate leakage around the outside of the valve (the latter known as paravalvular leakage or “PV” leakage). In the embodiment illustrated in FIGS. 1I-J, the cuff 260 only covers about half of the axial extent of stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g. wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. In some embodiments, the stent may be formed with cobalt chromium with additional metal or metal alloys such as nickel and/or molybdenum. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

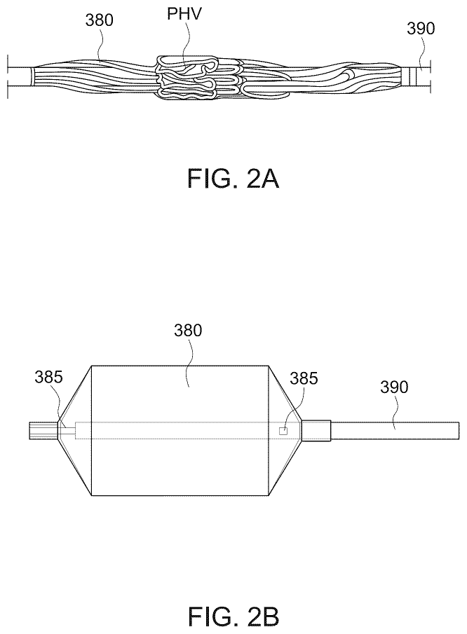

Referring to FIG. 2A, an example of a prosthetic heart valve PHV, which may include a stent similar to stents 100 or 200, is shown crimped over a balloon 380 of a balloon catheter 390 while the balloon 380 is in a deflated condition. It should be understood that other components of the delivery device, such as a handle used for steering and/or deployment, as well as a syringe for inflating the balloon 380, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 390 to inflate the balloon 380, as shown in FIG. 2B. FIG. 2B omits the prosthetic heart valve PHV, but it should be understood that, as the balloon 380 inflates, it forces the prosthetic heart valve PHV to expand into the native aortic valve annulus (although it should be understood that other heart valves may be replaced using the concepts described herein). In the illustrated example, fluid flows from a syringe (not shown) into the balloon 380 through a lumen within balloon catheter 390 and into one or more ports 385 located internal to the balloon 380. In the particular illustrated example of FIG. 2B, a first port 385 may be one or more apertures in a side wall of the balloon catheter 390, and a second port 385 may be the distal open end of the balloon catheter 390, which may terminate within the interior space of the balloon 380.

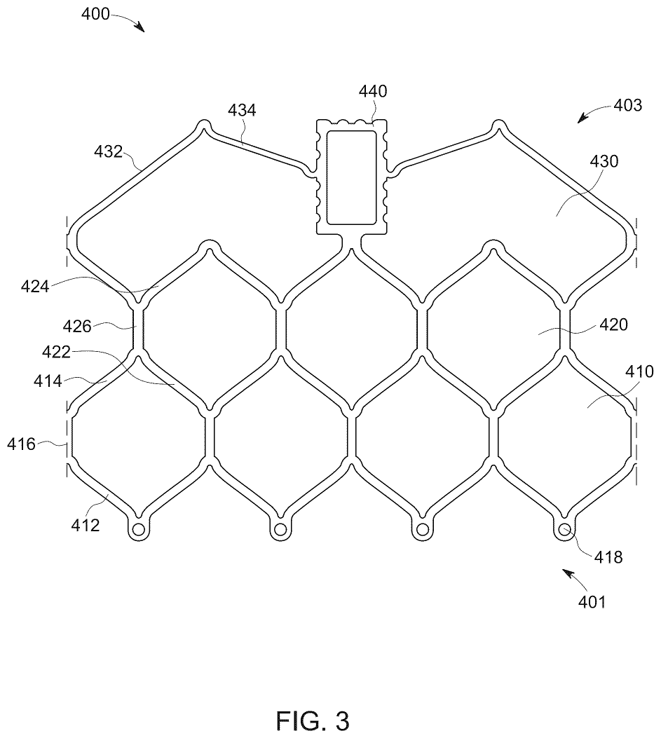

FIG. 3 is a plan view of a portion of a frame 400 according to another aspect of the disclosure, as if the frame has been cut longitudinally and laid flat on a table. Similar to FIGS. 1B-C, the portion of frame 400 shown in FIG. 3 is only a portion of the entire frame. If the frame 400 is used in a prosthetic heart valve that includes three prosthetic leaflets, the portion of frame 400 shown in FIG. 3 would be repeated two more times, and generally formed as a circumferentially continuous member, for a total of three iterations of the portion of frame 400 shown in FIG. 3. As with frames 100 and 200, frame 400 is preferably balloon-expandable and thus formed of a plastically-expandable material, such as cobalt chromium or stainless steel. Frame 400 may include an inflow end 401 and an outflow end 403.

Frame 400 may include an anchoring section toward the inflow end 401. In the illustrated embodiment, the anchoring section may be at least partially formed of two rows of generally diamond-shaped cells, including an inflow-most row 410 and an adjacent row 420. The cells in the inflow-most row 410 may be formed of struts, including two struts 412 that join to form an inflow-pointing apex, two struts 414 that join to form an outflow-pointing apex, and two longitudinal runners 416 that join the two apices. The cells of the adjacent row 420 may also be formed of struts, including two struts 422 that join to form an inflow-pointing apex, two struts 424 that join to form an outflow-pointing apex, and two longitudinal runners 426 that join the two apices. In the illustrated embodiment, struts 414 and struts 422 are shared among the two rows of cells. The anchoring section may be generally designed to help anchor the frame 400 into the native valve annulus, for example upon balloon-expansion of frame 400. Although frame 400 is illustrated with two rows of cells in the anchoring section, with the cells of each row being similar or identical to the other cells, it should be understood that many other specific embodiments of cells may be suitable for use in the anchoring section. For example, more than two rows may be used in the anchoring section, the cells of each row may have different shapes than other cells in the same row or other cells in other rows, the shapes of the cells may be other than that shown (e.g. hexagonal, octagonal, chevron-shaped), and the specific number and size of the cells may be different than that which his shown in FIG. 3. However, it is preferable that at least one cell in each section of the inflow-most row 410 includes a runner 416 with an axial extent for coupling to a radiopaque marker, as described in greater detail below.

Frame 400 may include a valve support section toward the outflow end 403. In the illustrated embodiment, the valve support section includes a row of hybrid cells 430 and a plurality of commissure attachment features (“CAFs”) 440. The inflow side of each hybrid cell 430 may be formed of four consecutive zig-zag struts 424 of cells in the second row 420. The remainder of each hybrid cell 430 may be formed of two outflow struts 432, 434 that join to form an outflow-pointing apex. A first end of strut 432 may couple directly to a strut 424, while a second end of strut 432 may couple to a first end of strut 434 to form the outflow-pointing apex. The second end of strut 434 may couple to a portion of a CAF 440, for example near an axial middle of one axial extension of CAF 440. In the illustrated embodiment, strut 432 has a width (in the inflow-to-outflow direction) that is greater than the width of strut 434. If the frame 400 is used in prosthetic heart valve, the frame 400 would preferably include a total of three CAFs 440, and two hybrid cells 430 between each circumferentially adjacent pair of CAFs 440. Although one particular embodiment of hybrid cell 430 is shown, it should be understood that other embodiments may be suitable. For example, three or more struts (instead of two struts 432, 434) may be included at the outflow side of the hybrid cells 430, the struts connecting to the CAFs 440 may connect at different locations than that shown, and the widths of the struts may be identical of different.

In the illustrated embodiment of frame 400, each CAF 440 is formed by four struts that together form a rectangular shape having a greater axial length that circumferential width. Also, in the illustrated embodiment, the outer surface of the two axial struts and the outflow-most strut include grooves, recesses, or divots. The CAFs 440 may function to assist with attaching commissures of the prosthetic leaflets to the frame 400. For example, the prosthetic heart valve that incorporates frame 400 may include three prosthetic leaflets (similar or identical to leaflets 250a-c), with each adjacent pair of leaflets being connected at a leaflet commissure, which is connected to the frame 400 via CAFs 440. In some examples, the prosthetic leaflet commissures can be coupled directly to the CAFs 440 (e.g. by suturing directly to the struts forming the CAFs 440), with the prosthetic leaflet commissures being positioned on the interior of the frame 400, or at least partially on the exterior by being pulled through the opening of the CAFs 440. In other examples, the prosthetic leaflet commissures can be indirectly attached to the CAFs 440, for example by attaching a fabric or tissue member to the CAFs 440 so that the fabric or tissue member spans the opening of the CAFs 440, and the prosthetic leaflet commissures may be directly attached (e.g. via suturing) to that fabric or tissue member. The grooves, recesses, or divots on the axial struts and the outflow-most strut (if included) may assist in securely maintaining the position of sutures that wrap around these struts. Although one particular embodiment of a CAF 440 is shown in FIG. 3, it should be understood that various other CAF designs may be just as suitable as the illustrated embodiment for use in coupling the prosthetic leaflet commissures to the frame 400.

In addition to prosthetic leaflets, the prosthetic heart valve the incorporates frame 400 may include other “soft” components, such as an interior cuff and/or exterior cuff, which may be similar, identical, or different than inner cuff 260 and outer cuff 270, respectively. In the illustrated embodiment of frame 400, the inflow apex of each cell in the inflow-most row 410 includes an aperture 418, which may receive sutures therethrough to assist in securing an inner cuff and/or outer cuff to the frame. However, in other embodiments, these apertures 418 may be omitted.

When deploying a transcatheter aortic valve replacement, including a prosthetic aortic valve that incorporates frames 100, 200 or 400, it is generally important to achieve proper deployment depth of the prosthetic heart valve relative to the native heart valve, and it is generally desirable for the rotational alignment of the prosthetic leaflet commissures to match the positioning of the native leaflet commissures. One way to assist with achieving desired deployment depth and/or commissure alignment is via imaging (e.g. using fluoroscopy) the prosthetic heart valve while it is positioned within the native heart valve prior to full expansion. Although metal frames are typically visible under fluoroscopy, it may be useful to include one or more radiopaque (“RO”) markers in or on the prosthetic valve to provide specific landmarks to assist with achieving recommended deployment depth and/or commissural position. It should be understood that, during a transcatheter aortic valve replacement procedure, more than one imaging modality may be used, for example fluoroscopy for imaging the prosthetic heart valve, and echocardiography for imaging the native tissue.

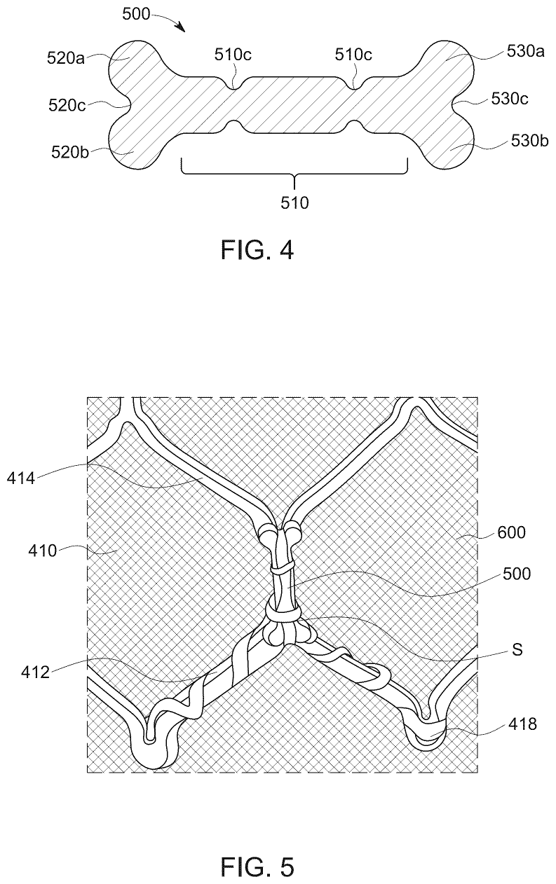

FIG. 4 is a front view of a RO marker 500 according to one aspect of the disclosure. RO marker 500 may be formed of a radiopaque material, such as a metal like tantalum, platinum, gold, or alloys thereof. Preferably, the material forming RO marker 500 is denser (and/or more radiopaque) than the material forming the frame of the prosthetic heart valve. In some examples, the RO marker 500 may by formed via laser cutting a sheet of the radiopaque material into the desired shape. In the illustrated example, RO marker 500 has a generally dog bone shape. For example, RO marker 500 may include an axial or longitudinal portion 510 that transitions at a first end to two angled protrusions 520a,b and at a second end to two angled protrusions 530a,b. In some embodiments, the angled protrusions may have a width that is about equal to the width of the axial portion 510, but extend only a short distance and end in a blunted (e.g. semicircular) shape. Recesses, grooves, or depressions 520c, 530c may be formed between each adjacent pair of angled protrusions 520a, 520b and 530a, 530b, respectively. In some embodiments, one or more recesses, grooves, or depressions 510c may be formed in the axial portion 510, the recesses extending inwardly toward the central longitudinal axis of the axial portion 510. In the illustrated example, four recesses 510 are provided in the axial portion 510, two on each side, but in other examples, more or fewer may be provided (in any desired configuration), or the recesses may be omitted. As explained in greater detail below, the recesses 510c, 520c, 530c, may provide areas in which sutures may reside to help better couple RO marker 500 to the frame of the prosthetic heart valve. In the illustrated embodiment, RO marker 500 is symmetric about both a longitudinal axis and a transverse axis (e.g., two planes of symmetry). However, in other embodiments, the RO marker 500 may be provided with an asymmetric shape/feature to help identify directionality under imaging (e.g., fewer than two planes of symmetry).

FIG. 5 is a perspective view of the RO marker 500 coupled to frame 400, with the frame 400 being in an expanded condition. In FIG. 5, an exemplary inner cuff 600 (which may be similar or identical to inner cuff 260) is shown radially inside of the frame 400. However, it should be understood that in actual use, the inner cuff 600 may have a different shape (e.g. the bottom or inflow end may more closely follow the inflow end of the frame 400), and may be coupled to the frame 400 different than shown in FIG. 5 (e.g. more sutures closely coupling the inner cuff 600 to the frame 400 along most or all of the struts in the first row 410 and/or second row 420 of cells, or coupled via other methods than sutures such as adhesives or ultrasonic welding).

As can be seen in FIG. 5, the profile of the RO marker 500 matches that of the frame 400 where it is attached to the frame 400. In particular, the axial portion of RO marker 500 generally aligns with a longitudinal runner 416, and the angled projections 520a,b and 530a,b generally align with small portions of corresponding struts 412, 414. In some embodiments, the angled projections 520a,b and 530a,b may extend at angles relative to the axial portion 510 that are substantially the same as the angles of struts 412, 414 relative to longitudinal runner 416 when the frame 400 is in the expanded condition.

In the illustrated embodiment, the RO marker 500 is coupled to a longitudinal runner 416 in the first row of cells 410 that axially aligns with a CAF 440. In other words, both the RO marker 500 and the corresponding CAF 440 are positioned at an identical angular location around the circumference of the frame.

In order to couple the RO marker 500 to the frame 400, the RO marker 500 is placed on a matching location on the frame 400, in this example along a longitudinal runner 416, such that the edges of the RO marker 500 align with the edges of the longitudinal runner 416. Similarly, the edges of the angled protrusions 520a,b and 530a,b may generally align with the edges of the corresponding struts 412, 414. While in this position, the RO marker 500 may be affixed to frame. In the illustrated example, the coupling is performed via wrapping one or more sutures S around the RO marker 500. For example, the suture S may be wrapped twice horizontally around the axial portion 510 of the RO marker 500, with the suture S sitting within recesses 510c, and once axially, with the suture S sitting within the recesses 520c, 530c. In the illustrated example, the horizontal suture wraps and vertical suture wrap extend around the longitudinal runner 416, and in some embodiments may also extend through the inner cuff 600. In other embodiments, the RO marker 500 may alternatively or additionally be coupled to the frame 400 via adhesives, welding, or lamination. If sutures are not used, one or more of the recesses 510c, 520c, 530c may be omitted. Furthermore, in some embodiments a non-metal buffer (e.g. fabric or polymeric buffer) may be positioned between the RO marker 500 and the frame 400 (e.g. by being sandwiched between the RO marker 500 and the longitudinal runner 416), to help avoid direct metal-to-metal contact and thus mitigate corrosion risk.

One benefit of the configuration of RO marker 500 and frame 400 shown in FIG. 5 is that the RO marker 500 is directly attached to the frame 400. It should be understood that, in this context, “direct” attachment includes the scenario in which a buffer is sandwiched between RO marker 500 and the frame 400, even if there is no direct metal-to-metal contact. Rather, the phrase “direct attachment” or “direct coupling” is meant to contrast to configurations in which an RO marker is coupled directly to a cuff, which is in turn coupled to the frame. The direct coupling of the RO marker 500 to the frame 400, such as the configuration shown in FIG. 5, may provide certain benefits over coupling an RO marker to a cuff which is coupled to the frame. One benefit is that due to the matching configuration between the RO marker 500 and the corresponding portions of the frame 400, there is little to no risk of incorrect placement of the RO marker 500. Compared to RO markers that are coupled to a cuff of a prosthetic heart valve, non-negligible ambiguity may be involved regarding the exact desired placement of the RO marker, particularly when there is not a corresponding matching shape that the RO marker can be matched to prior to coupling.

Additionally, the design of RO marker 500 and the corresponding portions of frame 400 may allow for direct attachment of the RO marker 500 to the frame 400 without impacting (or without meaningfully impacting) material properties of the frame 400. For example, prior art systems may provide a hole in the frame, with a RO rivet being placed within that hole. Although this type of rivet-in-hole design may provide for little or no ambiguity in where the RO rivet should be placed, this type of design impacts stress/strain of the frame local to the area of the RO rivet. In the configuration of FIG. 5, the area (e.g. longitudinal runner 416) the RO marker 500 attaches to does not bend or deform when the prosthetic heart valve is crimped or expanded, so the RO marker 500 has no more than minimal impact on structure and fatigue-resistance of the frame 400. In other words, the configuration shown and described in connection with FIG. 5 includes manufacturability benefits of a rivet-like concept, without the structural drawbacks of the rivet-like concept.

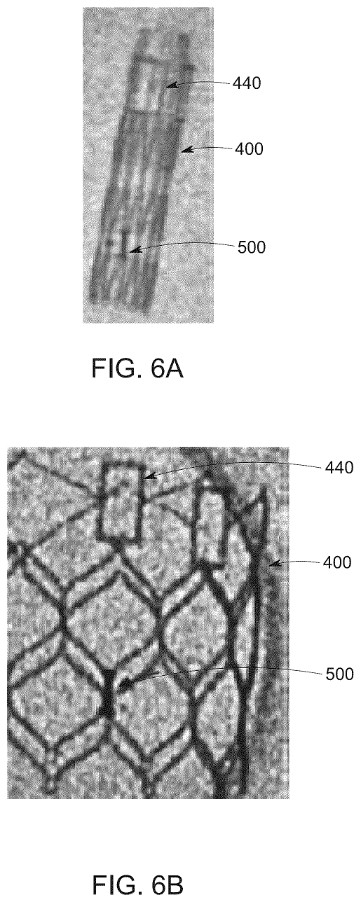

FIGS. 6A-B are fluoroscopic images of the RO marker 500 coupled to the frame 400, in the configuration shown in FIG. 5, while the frame 400 is in the crimped/collapsed and expanded conditions, respectively. As can be seen in both figures, the denser material of RO marker 500 helps to easily identify the location of the RO marker 500 compared to the frame 400, and the RO marker 500 is aligned axially with one of the CAFs 440. Further, the bottom or inflow end of the RO marker 500 will be generally aligned (although not perfectly) with the inflow end of the frame 400.

In use, a prosthetic heart valve that incorporates frame 400 (or a similar frame) may be manufactured with one RO marker 500 coupled to a longitudinal runner 416 in in the inflow-most row of cells 410, with the longitudinal runner 416 and RO marker 500 axially aligned with one of the CAFs 440. In some embodiments, more than one RO marker 500 may be used, for example a total of two, or a total of three (e.g. one for each CAF 440). The prosthetic heart valve may be crimped over a balloon of a delivery device, and with the balloon in a deflated condition, the delivery device may be passed through the patient's vasculature, for example through the femoral artery, around the aortic arch, and into the native aortic valve annulus. While the collapsed prosthetic heart valve is located within the native valve annulus, the prosthetic heart valve may be visualized, for example using fluoroscopy. The native tissue may also be visualized, for example using transesophageal echocardiography (“TEE”). Prior to fully inflating the balloon to fully deploy the prosthetic heart valve (e.g., while the balloon is entirely or only partially deflated), in some embodiments, the user may view the rotational position of the RO marker 500 relative to any one of the native leaflet commissures. In some examples, the imaging may be provided in multiple planes, and at least one plane may cross through one of the native leaflet commissures. To align the RO marker 500 with the native leaflet commissure, the user may rotate the prosthetic heart valve (e.g., via a knob on a handle of the delivery device) until the RO marker 500 is also within the same plane confronting the native leaflet commissure. As long as the RO marker 500 is rotationally aligned with any one of the three native leaflet commissures, all three of the CAFs 440 will be rotationally aligned with the three corresponding native commissures. In some embodiments, the RO marker 500 may also be used as a depth marker. In these embodiments, the bottom or inflow end of the RO marker 500 may be advanced to the desired depth within the native aortic valve annulus to achieve proper depth alignment prior to full expansion of the prosthetic heart valve. In some examples, the bottom or inflow end of the RO marker 500 may be generally aligned with the bottom or inflow end of the native aortic valve annulus while the balloon is fully deflated or partially deflated. Upon full expansion of the balloon and full deployment of the prosthetic heart valve, the inflow end of the prosthetic heart valve will be positioned at or near the inflow end of the native aortic valve annulus, with each of the prosthetic leaflet commissures rotationally aligning with each corresponding native leaflet commissure. In some embodiments, upon expansion, it may be preferable for the inflow edge of the prosthetic heart valve to be a few (e.g. 1, 2, or 3) millimeters below (in the ventricular direction) the native aortic valve. In some embodiments, the inflow edge of the frame does not shift axially during expansion. In these embodiments, the RO marker 500 may be used to help position the inflow edge of the frame about 3 mm below the annulus, and then the prosthetic heart valve may be expanded. In other embodiments in which the inflow edge of the prosthetic heart valve shifts in the outflow direction during expansion (e.g. due to effects of foreshortening), the prosthetic heart valve may be aligned prior to expansion deeper beyond the annulus to account for such potential foreshortening effects. It should be understood that the RO markers 500 may be used for either depth alignment, rotational alignment, or both.

In some embodiments, due to folding of the balloon, the balloon itself may rotate or “unfold” as it expands. In these cases, as the balloon begins to unfold, it may cause the prosthetic heart valve to rotate during expansion. For example, during the first 10-30% of balloon expansion, the prosthetic heart valve may rotate a given amount (e.g. 15 degrees, 30 degrees, 45 degrees, 60 degrees, etc.). In such embodiments, it may be preferable to align the RO markers 500 not with the CAFs 440, but rather offset from the CAFs 440 by a known distance that corresponds to this initial rotation. In these embodiments, the RO markers 500 may be aligned with the native commissures prior to beginning balloon expansion. At this stage, the CAFs 440 would be intentionally offset rotationally form the native commissures. As the balloon begins to expand, it may unfold and cause the prosthetic heart valve to rotate the known amount. Because of the above-described offset, upon completing rotation due to balloon unfolding, the CAFs 440 may be substantially aligned with the native commissures, even though after expansion, the CAFs 440 are no longer aligned with the native commissures.

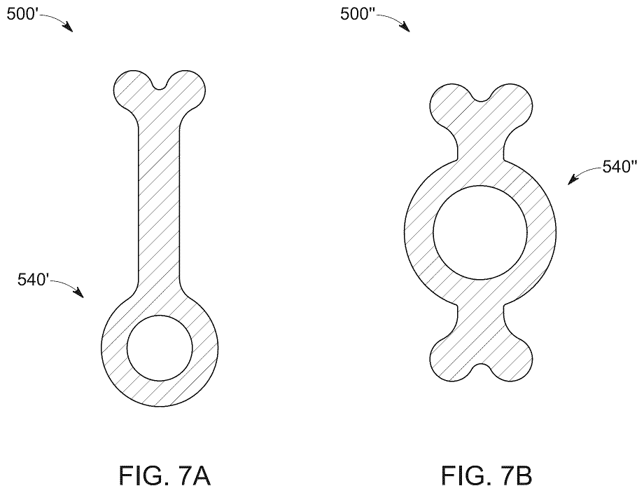

Although FIG. 4 illustrates one embodiment of RO marker 500, other embodiments may include additional features to help better mark the depth of the prosthetic heart valve. For example, FIGS. 7A and 7B illustrate RO markers 500′, 500″, that are identical to RO marker 500 with the exception of an additional height marker. In FIGS. 7A-B, the additional height marker is shown in the form of a ring 540′, 540″, which may be formed integrally with the remainder of RO marker 500′, 500″, or separately attached thereto. Preferably, the rings 540′, 540″ are formed of the same material as the RO markers 500′, 500″. Each ring 540′, 540″ may take the form of an annulus, with an open interior. In FIG. 7A, ring 540′ is positioned so that the top of the inner opening is aligned with the bottom of the RO marker 500′, with the remainder of the ring 540′ extending in the inflow direction from the RO marker 500′. In FIG. 7B, the ring 540″ is positioned so that the center of the inner opening is aligned with the center of the axial portion of the RO marker 500″. If ring 540′ or 540″ is included, during (or prior to) deployment of the prosthetic heart valve, depth may be measured from the bottom or inflow end of the stent to the top or bottom of the inner opening of the ring 540′ or 540″. For example, RO marker 500 shown in FIGS. 4-5 nearly perfectly matches features of the frame, which may limit the actual depth that can be marked. However, for the markers 500′ (with a ring 540′ outside the marker) and 500″ (with a ring 540″ inside the marker), the inside of the circle of the ring can be lined up with the annulus to achieve the desired depth, which may provide for additional flexibility and/or precision in terms of how the depth is being marked.

It should be understood that the RO markers 500, 500′, 500″ shown in FIGS. 4 and 7A-B, are just one option for a shape of the RO markers. The shape of the RO markers may be modified, depending on the shape of the frame, including the shape of the struts of the frame, where the RO markers will be attached. For example, markers 500′, 500″ may include notches similar to marker 500. Furthermore, frames 100, 200, and 400 are only some examples of frames with which RO markers may be used.

In some embodiments, the RO markers may be provided integrally with the frame. For example, instead of providing RO marker 500 as a separate member at the location shown in FIG. 5, the vertical strut to which the RO marker 500 would be attached could be provided with increased thickness (thickness being measured in the depth direction between the outer and inner surfaces of the frame). This could be achieved, for example, by forming the frame from a tube having a constant thickness, and then shaving or otherwise reducing the thickness of the frame in areas that are not meant to serve as a RO marker by having increased frame thickness. In these embodiments, the methodology for depth alignment and/or commissure alignment may be the same as described above in connection with RO marker 500, but a separate step of attaching the RO marker 500 to the frame may be omitted.

Furthermore, although the RO markers described above are described in the context of a balloon-expandable prosthetic heart valve, it should be understood that the concepts generally apply with equal force to self-expanding prosthetic heart valves. In other words, RO markers similar to those described above may be formed with stent-matched shapes, but with the stent being a self-expanding shape (e.g. formed of shape memory alloys such as nitinol).

Still further, as described above, in some embodiments a single RO marker may be provided on the frame, and in other embodiments multiple RO markers may be provided on the frame. If multiple RO markers are provided on the frame, they may all be of the same construction, or at least some (including all) may be of different construction. For example, a frame may be provided with the RO marker 500 shown in FIG. 4 aligned with one CAF 440, the RO marker 500′ shown in FIG. 7A aligned with a second CAF 440, and the RO marker 500″ shown in FIG. 7B aligned with a third CAF 440. This is just one example, and it should be understood that other combinations may be suitable. If different or unique RO markers are provided on the same frame, the RO markers may help to better understand what portion of the frame is in the foreground and what portion of the frame is in the background on the imaging, as such information is not always immediately clear. In some embodiments, the prosthetic heart valve may not be rotationally symmetric. For example, a frangible portion may be provided in one area of the frame to assist in a future valve-in-valve implantation in which the original frame is broken, fractured, or otherwise manipulated to increase the space available for a second valve implantation. In other examples, the frame may include cut-outs or other unique shapes to help ensure that impingement on conduction centers are minimized, with the cut-outs only being located at one or certain locations that result in rotational asymmetry. In still other examples, the prosthetic valve assembly within the frame may include asymmetric leaflets which are not easily identifiable under fluoroscopy. In these examples, the use of uniquely shaped RO markers around the circumference of the frame may help with better understanding the rotational position of the frame relative to the anatomy.

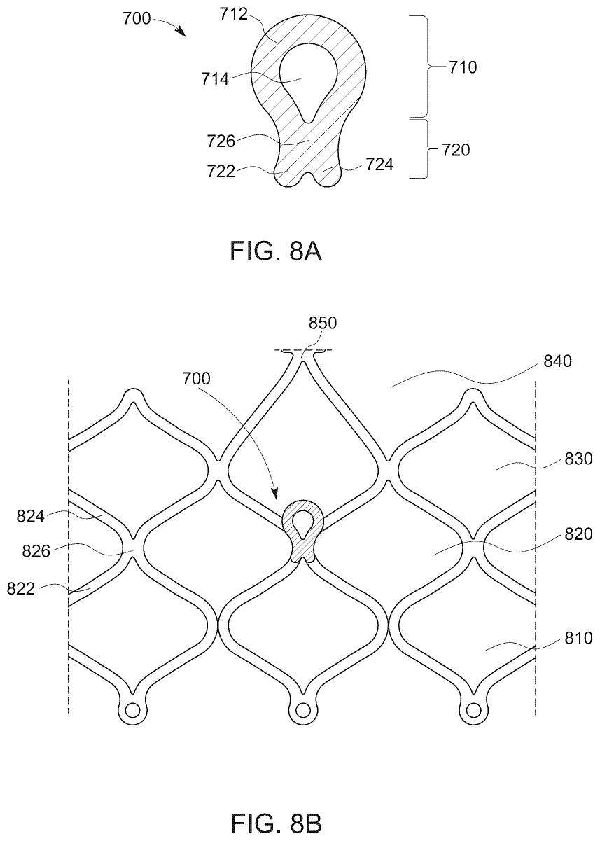

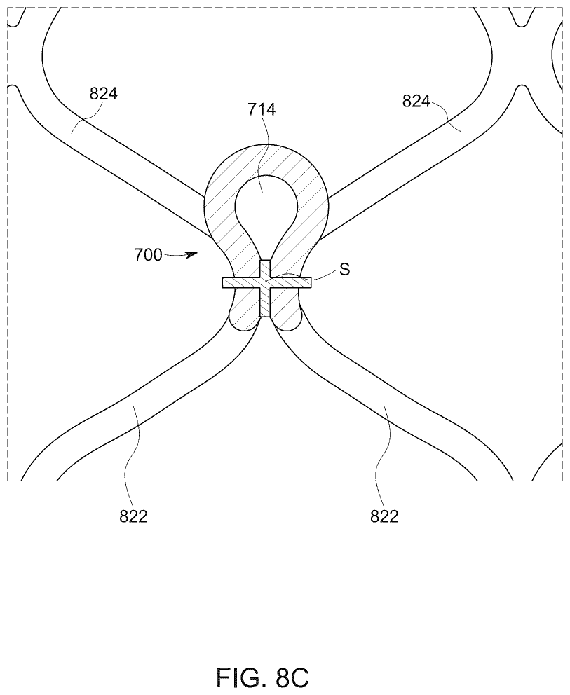

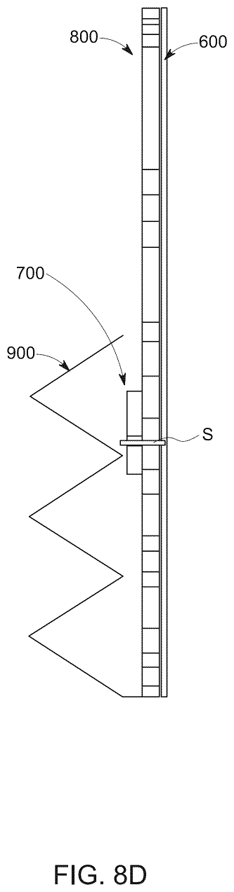

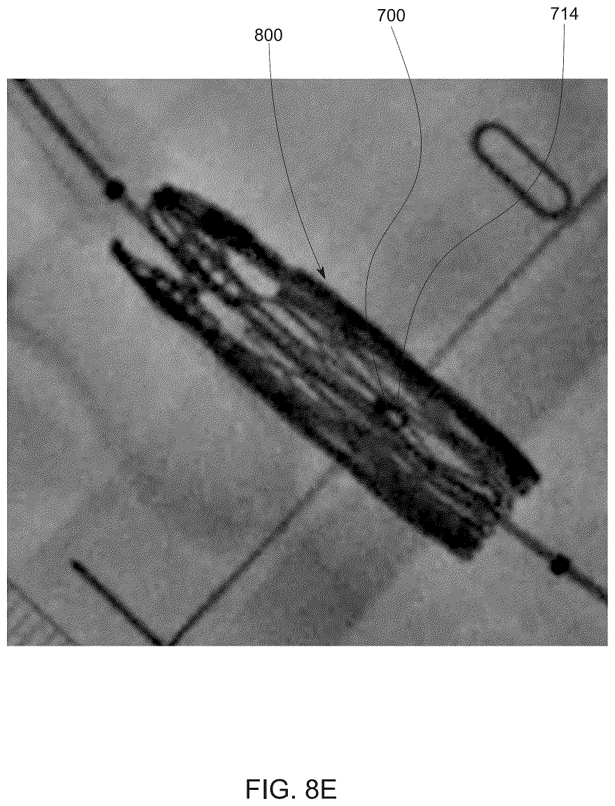

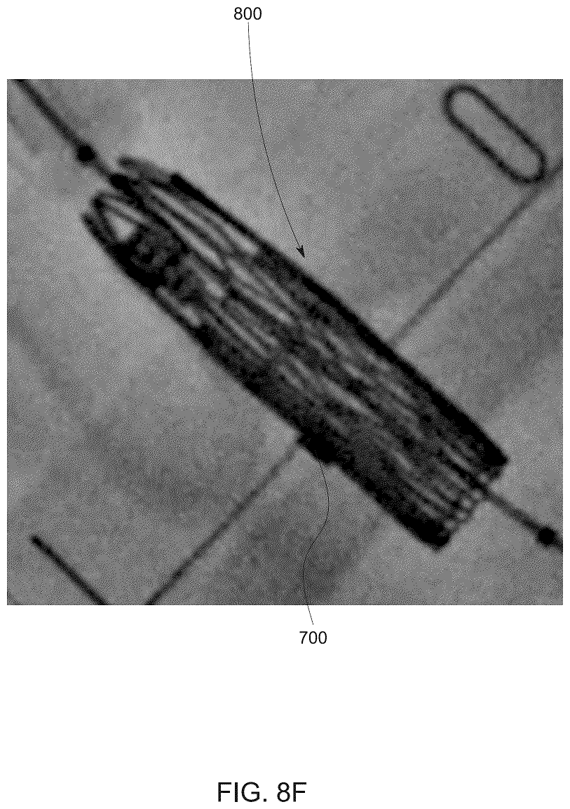

FIG. 8A is a front view of another example of a RO marker 700 according to an aspect of the disclosure. The RO marker 700 may be formed from any of the materials described above in connection with the other RO markers described herein, and may have the same general purpose as the other RO markers described herein, although while having certain other features. In the illustrated embodiment, RO marker 700 has two substantially flat sides, one of which is visible in FIG. 8A, although instead of being flat, the opposing surfaces may have a small amount of curvature (e.g. one slightly convex side, one slightly concave side) to match the slight contour of an outer surface of a frame to which the RO marker 700 is coupled.

Generally, RO marker 700 includes a first or top portion 710 which may include a loop 712 that is generally circular or oval or tear drop-shaped, such that a corresponding window or opening 714 is formed within the loop 712, the opening being generally circular or oval or tear drop-shaped. The RO marker 700 may include a second or bottom portion 720 formed of two short legs 722, 724 that meet at an intersection 726. As should be understood from the description below, the two short legs 722, 724 and intersection 726 may generally match the shape and contour of portions of two struts (and the intersection of those two struts) of the frame of the prosthetic heart valve that incorporates the RO marker 700.

FIG. 8B is a plan view of an example of a portion of a frame 800 of a prosthetic heart valve to which the RO marker 700 is attached. As with frame 400 of FIG. 3, FIG. 8B only shows a portion of the frame 800, as if the frame has been cut longitudinally and laid flat on a table. Frame 800 may be substantially similar or identical to frame 400, and thus the description above in connection with frame 400 generally applies to frame 800, although some differences are illustrated. It should be understood that, although RO marker 700 is shown and described in connection with frame 800, as with the other RO markers described herein, RO marker 700 may be used with other frames that are not identical to those explicitly shown and described herein. While frame 400 is illustrated with an inflow row 410 of cells, an adjacent row 420 of cells, and a hybrid row 430 of cells, frame 800 is illustrated with an inflow row 810 of cells, a middle row 820 of cells, an outflow row of cells 830. Frame 800 may include a hybrid row 840 of cells above the outflow row 830 of cells, which are only partially visible in FIG. 8B. However, it should be understood that the hybrid row 840 of cells may be similar or identical to hybrid row 430 of frame 400, and thus additional description or illustration is unnecessary. As with frames 100, 200, and 400, frame 800 is preferably balloon-expandable and thus formed of a plastically-expandable material, such as cobalt chromium or stainless steel.

Each cell in the inflow row 810, the middle row 820, and the outflow row 830 may be formed by four struts, including two inflow struts and two outflow struts. As an example, each cell in the middle row 820 of cells may include two inflow struts 822 forming a general zig-zag pattern and two outflow struts 824 forming a generally zig-zag pattern, so that a generally diamond-shaped cell is formed. Where two adjacent cells each row meet, four struts may form an intersection. For example, where adjacent inflow struts 822 of adjacent cells meet, which is also where adjacent outflow struts 824 of adjacent cells meet in the middle row 820, an intersection 826 may be formed. The shape formed by the intersection 826 and the portions of struts 822, 824 extending therefrom may generally match the shape of the lower portion 720 of the RO marker 700. In the illustrated embodiment, RO marker 700 is coupled to the frame 800 at an intersection of two struts in the middle row 820, with the RO marker 700 being positioned in longitudinal alignment with a CAF 850. Although CAF 850 is not fully shown in FIG. 8B, CAF 850 may be substantially similar or identical in function and structure to CAF 440 shown in FIG. 3 and described in greater detail above.

FIG. 8C is an enlarged view of an example of RO marker 700 attached to frame 800, at the intersection of two outflow struts 824 and two inflow struts 822. In the illustrated embodiment, one or more sutures S are wrapped around both the RO marker 700 and the strut intersection, for example including a horizontal wrap and a vertical wrap, with the vertical wrap passing through the opening 714 of the RO marker 700. However, various other connection modalities to couple the RO marker 700 to the frame 800 may be suitable. Although FIGS. 8B-C show a frame 800 with three rows of cells (not including the hybrid row of cells) and the RO marker 700 being coupled between two adjacent cells in the middle row 820 of cells, in other examples, the frame may include two rows of cells (not including the hybrid row of cells) and the RO marker could be coupled between two adjacent cells in either the inflow row of the two rows, or the outflow row of the two rows. And although FIGS. 8B-C show the RO marker 700 coupled to two adjacent cells in the middle row 820 of cells, in other embodiments, the RO marker 700 could be coupled to two adjacent cells in the inflow row 810 or in the outflow row 830.

FIG. 8D shows a side view of frame 800 with additional components of a prosthetic heart valve. For example, in FIG. 8D, the radially inner surface of the frame 800 is to the right in the view of FIG. 8D while the radially outer surface of the frame 800 is to the left in the view of FIG. 8D. FIG. 8D shows that an inner cuff 600 (which may be similar or identical to inner cuff 600 described above, but which may also take other forms) may be positioned (and/or coupled) the radially inner surface of the frame 800. Similarly, an outer cuff 900 may be positioned (and/or coupled) on a radially outer surface of frame 800. In the particular illustrated example, the inner cuff 600 extends from the inflow end of the frame 800 toward the outflow end of the frame 800 (although not necessarily up to the end of the hybrid cells), with the inner cuff 600 generally lying flat on the inner surface of the frame 800. Also in this particular illustrated example, the outer cuff 900 forms undulating pleats and extends from the inflow end of the frame 800 to a point above the RO marker 700. With this configuration, the RO marker 700 is positioned between the inner skirt 600 and outer skirt 900. However, it should be understood that the inner skirt 600 and outer skirt 900 are only exemplary, and a prosthetic heart valve that incorporates a frame and RO marker 700 may be provided with or without inner and/or outer skirts, and if such skirts are provided, they may take other forms than shown herein. It should also be understood that a prosthetic heart valve incorporating the components of FIG. 8D would typically include three prosthetic leaflets (although more or fewer leaflets may be provided), but the prosthetic leaflets are omitted from FIG. 8D for clarity.

FIG. 8E is a radiograph of the RO marker 700 of FIG. 8A coupled to the frame 800 of FIG. 8B, with the radiograph showing the frame 800 at a first rotational angle. As can be seen in FIG. 8E, the RO marker 700 appears darker than the frame 800 on the radiograph, due to the RO marker 700 being formed of a denser (and/or more radiopaque) material than the frame 800. Further, due to the rotational orientation of the frame 800 relative to the imager that created the radiograph, the opening 714 is clearly visible in the radiograph, providing a type of “target” (or multiple targets) that allows the user to easily understand from the image where the RO marker 700 is (and thus where the remaining portions of the frame 800 are) relative to other objects in the radiograph. In FIG. 8E, the window 714 may be largely or entirely unobstructed by other adjacent portions of the frame 800, including the portion(s) of the frame to which the RO marker 700 is coupled. FIG. 8F shows a radiograph taken after the frame 800 has been rotated about 90 degrees. Due to the orientation of the RO marker 700 relative to the imaging device, the opening 714 of the RO marker 700 is not visible, but the RO marker 700 is highly visible in the radiograph, not only because it is formed of a relatively dense material, but also because it protrudes radially outwardly form the otherwise cylindrical profile of the collapsed frame 800.

The RO marker 700 may be used in substantially the same fashion as described in connection with other embodiments of RO markers described herein, including for example to achieved a desired depth alignment of the prosthetic heart valve with the native valve annulus, and/or achieving rotational alignment between the prosthetic leaflet commissures (and thus CAFs of the frame) with that native commissures of the native heart valve (or with prosthetic commissures of a prosthetic heart valve if this is a valve-in-valve procedure in which a prosthetic heart valve is being used to replace a previously-implanted prosthetic heart valve).

For all of the embodiments of RO markers described herein, including RO marker 700, the RO marker may also be used in a slightly different way depending on the use range of the prosthetic heart valve incorporating the RO marker. For example, a prosthetic heart valve that incorporates frame 800 and RO marker 700 may be adapted to be implanted in a native aortic valve having a range of sizes. As one example, such a prosthetic heart valve may be provided in multiple nominal sizes, for example a 29 mm nominal size that is configured for implantation into a native annulus that is as small as 26 mm in diameter or as large as 30 mm in diameter. If that prosthetic heart valve is implanted into a patient with a 26 mm diameter annulus, the prosthetic heart valve may be considered within or at the “low-use range” (e.g. at the minimum deployed valve diameter) whereas if that prosthetic heart valve is implanted into a patient with a 30 mm diameter annulus, the prosthetic heart valve may be considered within or at the “high-use range” (e.g. at the maximum deployed valve diameter). In both situations, the same prosthesis is being implanted, but the prosthesis is being expanded to a different diameter to suit the needs of the specific patient. The total amount of expansion may be related to the resulting axial height of the prosthesis, since the prosthesis generally foreshortens as it radially expands. In other words, the intended diameter to which the prosthetic heart valve is expanded during deployment may affect the final resulting axial depth of the prosthetic heart valve relative to the axial depth of the prosthetic heart valve immediately before deployment (while it remains collapsed over a balloon of the delivery device). Stated in another way, the initial depth positioning of the prosthetic heart valve just prior to expansion into the patient's annulus may need to be adjusted depending on whether the prosthetic heart valve is being used within the low-use range or the high-use range (or anywhere in between).

Because one particular nominally sized prosthetic heart valve may be adapted for use in a variety of patients with a different resulting expanded diameter of the frame, it would be useful if a single RO marker could be used to achieve desired axial depth alignment for different expected implantation diameters of the prosthetic heart valve. Any of the RO markers described herein may be used with multiple targets (which may or may not be separate structural features, such as indentations, contours, etc.) to use during depth alignment, with the particular target being used for the depth alignment corresponding to the expected final diameter of the expanded prosthetic heart valve. One particular example of this is described in connection with RO marker 700. If a prosthetic heart valve incorporating a frame (e.g. frame 800) and RO marker 700 is being used in the prosthetic heart valve's low-use range, while the prosthetic heart valve remains collapsed and is being imaged (e.g. under fluoroscopy), the user may align the center of the circular opening 714 with the plane of the patient's valve annulus just prior to deployment. However, if that same prosthetic heart valve is being used in the prosthetic heart valve's high-use range, while the prosthetic heart valve remains collapsed and is being imaged (e.g. under fluoroscopy), the user may align the top of the circular opening 714 with the plane of the patient's valve annulus just prior to deployment. In other words, different portions of the RO marker 700 (e.g. different targets) may be aligned with the same anatomical landmark (e.g. the annular plane), so that the valve is positioned a little deeper just prior to deployment when used in the high-use range, or a little shallower just prior to deployment in the low-use range, as this initial depth differential will be compensated for the different amount of foreshortening resulting from the different expansion diameter.

It should be understood that the differential targeting of the RO marker relative to the patient anatomical landmarks based on expected use range of the prosthetic heart valve provided above is merely exemplary. In other words, depending on the specific shape and design of the RO marker, and where it is coupled to the relevant frame, the desired target for a particular use range may be different than that provided above. For example, with the generally dog bone-shaped RO marker 500, the different depressions 510c or different ends of the dog bone shape may be used as the relevant target depending on the expected final expanded diameter of the prosthetic heart valve.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve, comprising:
an expandable frame extending along a longitudinal axis between an inflow end and an outflow end, the frame having a plurality of cells, each of the plurality of cells formed at least by two inflow struts and two outflow struts, wherein two circumferentially adjacent cells connect at a strut intersection member where two of the inflow struts of the two circumferentially adjacent cells connect to two of the outflow struts of the two circumferentially adjacent cells, the frame including a plurality of commissure attachment features, a first one of the plurality of commissure attachment features being axially aligned with the strut intersection member of the two circumferentially adjacent cells;
a plurality of prosthetic leaflets mounted within the frame, a first pair of the plurality of prosthetic leaflets being coupled together to form a first prosthetic commissure, the first prosthetic commissure being attached to the first one of the plurality of commissure attachment features; and
a radiopaque marker having a first portion and a second portion, the first portion of the radiopaque marker defining a window, the second portion of the radiopaque marker being coupled to the strut intersection member of the two circumferentially adjacent cells so that so that the window and the second portion is aligned with the strut intersection member of the two circumferentially adjacent cells, the radiopaque marker being non-integral with the frame.

2. The prosthetic heart valve of claim 1, wherein the window and the second portion of the radiopaque marker is also aligned with the first one of the plurality of commissure attachment features.

3. The prosthetic heart valve of claim 1, wherein the plurality of cells includes a first inflow row of cells, a second middle row of cells, and a third outflow row of cells, the two circumferentially adjacent cells being part of the second middle row of cells.

4. The prosthetic heart valve of claim 1, wherein the plurality of cells includes a first inflow row of cells and a second outflow row of cells, the two circumferentially adjacent cells being part of the first inflow row of cells.

5. The prosthetic heart valve of claim 1, wherein the plurality of prosthetic leaflets comprises exactly three prosthetic leaflets, and the plurality of commissure attachment features comprises exactly three commissure attachment features.

6. The prosthetic heart valve of claim 5, wherein a second pair of the plurality of prosthetic leaflets are coupled together to form a second prosthetic commissure attached to a second one of the plurality of commissure attachment features, and a third pair of the plurality of prosthetic leaflets are coupled together to form a third prosthetic commissure attached to a third one of the plurality of commissure attachment features.

7. The prosthetic heart valve of claim 6, wherein the radiopaque marker is the only radiopaque marker coupled to the prosthetic heart valve.

8. The prosthetic heart valve of claim 1, wherein the frame is formed of stainless steel or cobalt chromium, and the radiopaque marker is formed of gold, platinum, or tantalum.

9. The prosthetic heart valve of claim 1, wherein the window is circular or tear drop-shaped, and in an expanded condition of the frame, the window is radially aligned with an empty space defined by a selected one of the plurality of cells.

10. The prosthetic heart valve of claim 1, wherein the first portion of the radiopaque marker has a first shape that at least partially matches a shape of the two of the outflow struts of the two circumferentially adjacent cells, and the second portion of the radiopaque marker has a second shape that at least partially matches a shape of the two of the inflow struts of the two circumferentially adjacent cells.

11. The prosthetic heart valve of claim 1, further comprising an inner cuff on an interior surface of the frame, and an outer cuff on an exterior surface of the frame.

12. The prosthetic heart valve of claim 11, wherein the radiopaque marker is positioned between the inner cuff and the outer cuff.

13. The prosthetic heart valve of claim 12, wherein the inner cuff and the outer cuff are each formed of fabric.

14. The prosthetic heart valve of claim 1, wherein the second portion of the radiopaque marker is coupled to the strut intersection member by one or more sutures having a suture pattern.

15. The prosthetic heart valve of claim 14, wherein the suture pattern includes a horizontal portion that wraps around the second portion of the radiopaque marker and the strut intersection member.

16. The prosthetic heart valve of claim 15, wherein the suture pattern includes a vertical portion that wraps around the second portion of the radiopaque marker and the strut intersection member, the vertical portion passing through the window.

17. A method of implanting a prosthetic heart valve, comprising:
advancing the prosthetic heart valve through a vasculature of a patient while the prosthetic heart valve is in a collapsed condition until the prosthetic heart valve is positioned within or adjacent to a native heart valve annulus of the patient, the prosthetic heart valve including a frame defining a plurality of cells and being formed of a metal or metal alloy having a first radiopacity, a radiopaque marker being coupled to the frame, the radiopaque marker having a second radiopacity greater than the first radiopacity;
while the prosthetic heart valve is positioned within or adjacent to the native heart valve annulus of the patient, viewing an image of the prosthetic heart valve positioned within or adjacent to the native heart valve annulus of the patient to determine a position of a target on the radiopaque marker relative to an anatomical landmark in the image; and
deploying the prosthetic heart valve by expanding the prosthetic heart valve into the native heart valve annulus,
wherein the step of deploying the prosthetic heart valve is performed only after determining that the target on the radiopaque marker is aligned with the anatomical landmark in the image,
wherein the target on the radiopaque marker is selected from a plurality of targets on the radiopaque marker based on the expected diameter that the prosthetic heart valve will have after expanding the prosthetic heart valve into the native heart valve annulus.

18. The method of claim 17, wherein the radiopaque marker includes a first portion and a second portion, the first portion of the radiopaque marker defining a window, the second portion of the radiopaque marker being coupled to a portion of the frame.

19. The method of claim 18, wherein the plurality of targets on the radiopaque marker include (i) a center of the window and (ii) another location within the window spaced from the center of the window.

20. The method of claim 19, wherein the anatomical landmark is a plane of the native heart valve annulus, and while viewing the image of the prosthetic heart valve, at least a portion of the window is unobstructed by the portion of the frame to which the second portion of the radiopaque marker is coupled.
